(19) United States
(12) Patent Application Publication
(10) Pub. No.: US 2018/0365877 A1
(43) Pub. Date: Dec. 20, 2018

(54) SYSTEMS FOR ADAPTIVE CONTROL DRIVEN AR/VR VISUAL AIDS

(71) Applicant: EYEDAPTIC, LLC, LAGUNA NIGUEL, CA (US)

(72) Inventors: David A. Watola, Irvine, CA (US); Jay E. Cormier, Laguna Niguel, CA (US); Brian Kim, San Clemente, CA (US)

(21) Appl. No.: 15/918,884

(22) Filed: Mar. 12, 2018

Related U.S. Application Data
(60) Provisional application No. 62/470,297, filed on Mar. 12, 2017.

(51) Int. Cl. G06T 11/60; G06T 3/40; G06T 5/00; G02B 27/01; A61F 9/08

(52) U.S. Cl. CPC G06T 11/60; G06T 3/40; G06T 5/006; G02B 27/0172; G02B 2027/011; G06T 2200/24; G02B 2027/0178; G02B 2027/014; G02B 2027/0138; A61F 9/08

(57) ABSTRACT

Interactive systems using adaptive control software and hardware from known and later developed eye-pieces to later developed head-wear to lenses, including implantable, temporarily insert-able and contact and related film based types of lenses including thin film transparent elements for housing cameras lenses and projector and functional equivalent processing tools.

### CROSS REFERENCE TO RELATED CASES

This application claims the benefit of and priority to U.S. Provisional Patent Application Ser. No. 62/470,297, filed on Mar. 12, 2017, the content of which is incorporated herein by reference herein in its entirety. Likewise, expressly incorporated by reference, as if fully set forth herein are commonly owned and assigned U.S. Ser. Nos. 62/530,286; 62/530,792; 62/579,657; 62/579,798; and PCT/US17/62421 along with U.S. Ser. No. 15/817,117.

### FIELD

The present disclosures relate to the art and science of visual field characterization, diagnosis and amelioration by devices, drugs and artificially intelligent homunculi, along with known, in process and later developed related image replacement morphologies.

The present inventions likewise are on knowledge, ensconced within the forefront of the cutting edge of Augmented and Virtual Reality Houseware/firmware/hardware, software and linking elements, and chimeric or hybridized versions of the same.

This section is very brief, with the next major section immediately presenting technical information related to image processing and warping functions. Known prior art focuses primarily on the simple operations of magnification and contrast enhancement for low-vision users. The ideas presented here are considerably more complex. The present invention improved in kind, not just degree, it is respectfully proposed, over prior art. The flowchart for warping and mapping methodologies are as follows:

### BACKGROUND OF THE DISCLOSURES

The Interactive Augmented Reality (AR) Visual Aid invention described below is intended for users with visual impairments that impact field of vision (FOV). These may take the form of age-related macular degeneration, retinitis pigmentosa, diabetic retinopathy, Stargardt's disease, and other diseases where damage to part of the retina impairs vision. The invention described is novel because it not only supplies algorithms to enhance vision, but also provides simple but powerful controls and a structured process that allows the user to adjust those algorithms.

The basic hardware is constructed from a non-invasive, wearable electronics-based AR eyeglass system (see figure) employing any of a variety of integrated display technologies, including LCD, OLED, or direct retinal projection. One or more cameras, mounted on the glasses, continuously monitor the view where the glasses are pointing. The AR system also contains an integrated processor and memory storage (either embedded in the glasses, or tethered by a cable) with embedded software implementing real time algorithms that modify the images as they are captured by the camera(s). These modified or corrected images are then continuously presented to the eyes of the user via the integrated displays.

The basic image modification algorithms come in multiple forms as described later, in conjunction with the AR hardware glasses, they enable users to enhance vision in ways extending far beyond simple image changes such as magnification or contrast enhancement. The fundamental invention is a series of adjustments that are applied to move, modify; or reshape the image in order to reconstruct it to suit each specific user's FOV and take full advantage of the remaining useful retinal area. The following disclosure describes a variety of mapping, warping, distorting and scaling functions used to correct the image for the end user.

The invention places these fundamental algorithms under human control, allowing the user to interact directly with the corrected image and tailor its appearance for their particular condition or specific use case (see flowchart below). In prior art, an accurate map of the usable user FOV is a required starting point that must be known in order to provide a template for modifying the visible image. With this disclosure, such a detailed starting point derived from FOV measurements does not have to be supplied. Instead, an internal model of the FOV is developed, beginning with the display of a generic template or a shape that is believed to roughly match the type of visual impairment of the user. From this simple starting point the user adjusts the shape and size of the displayed visual abnormality, using the simple control interface to add detail progressively, until the user can visually confirm that the displayed model captures the nuances of his or her personal visual field. Using this unique method, accurate FOV tests and initial templates are not required. Furthermore, the structured process, which incrementally increases model detail, makes the choice of initial model non critical.

### OBJECTS AND SUMMARY OF THE INVENTION

Briefly stated, Interactive systems using adaptive control software and hardware from known and later developed eye-pieces to later developed head-wear to lenses, including implantable, temporarily insert-able and contact and related film based types of lenses including thin film transparent elements for housing cameras lenses and projector and functional equivalent processing tools.

According to embodiments there is disclosed wearable electronic head-mounted augmented reality (AR) glasses low vision aid comprising, in combination. At least one embedded video camera situated and configured for capturing real-time imagery that encompasses at least the field-of-view of a normally-sighted human wearer; an embedded display or displays, each presenting imagery directly to one of the wear's eyes said imagery originating from said embedded camera(s) said imagery processed, enhanced, or manipulated arbitrarily according to the user's need or benefit, including magnification and nonlinear transformations, said display(s) arranged such that their contents will be centered upon the macula of the retina of the associated eye, where the highest visual acuity would be available to a normally-sighted wearer; at least a computational processing device, embedded or remote, for producing processed said images for said display(s) from camera images; a means for controlling the specific computations performed by said computational device, including physical control surfaces, remote-control devices, voice control, or autonomous software-based decision; and, a physical barrier, placed explicitly or existing implicitly in the construction of the display, that prevents external scene light (especially light originating directly behind the apparent positon of the displays) from impinging on the same portions of the retina as imagery from the displays, said barrier eliminating focus ambiguities that confound low-vision users, whereby unobstructed pathways for external scene light to enter the eye(s) and impinge on the retina directly around the periphery (side and preferably top and bottom) of the displayed image(s) at the natural retinal focus (as if no glasses were present for said rays of external scene light), said unobstructed pathways providing failsafe vision if display devices fail to function, and said unobstructed pathways providing a zero-latency reference for equilibrium.

According to embodiments there is disclosed, a wearable electronic head-mounted augmented reality (AR) glasses low vision aid, further comprising additional processing and computations (using the computational processing device) upon displayed image(s) such that the boundary between natural scene lighting and displayed imager upon the retina can appear natural and seamless when interpreted by the wearer's brain; said natural-seeming transition maintaining even when arbitrary non-natural processing is applied near the center of the displayed image(s).

According to embodiments there is disclosed, a wearable electronic head-mounted augmented reality (AR) glasses low vision aid, further comprising output providing an undiminished, naturally-wide overall field-of-view limited only by the physical superstructure of the glasses themselves,, even if the active display field-of-view is much smaller.

### BRIEF DESCRIPTION OF THE DRAWINGS

Various preferred embodiments are described herein with references to the drawings in which merely illustrative views are offered for consideration, whereby:

FIG. 1 is a schematic depiction of contact lenses; glasses and standard AR/VR headgear including any known or later developed interchangeable hardware component;

FIG. 2 is a technical problem solving explanation showing data-flow according to the teachings of the present disclosures whereby embodiments of the instant teachings driven by the instant software cure visual deficits and deficiencies using AR/VR modalities; and

FIG. 3 is a schematic of flow-chart or logic sequence, for example for any data used with an exemplary embodiment, for example the large framed-style of glasses shown in FIG. 1, and all of the interfaces with technology components.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTIONS

The present inventors have created software and user interface improvements taking AR/VR to the next level for vision-impaired users, each of the following references, defining the state of the art is fully incorporated herein by reference as are US 20160270648, U.S. Pat No. 9,516,283 EP2674805, CA2820241, U.S. Pat. No. 8,976,086, U.S. Pat. No. 9,372,348, US20160282628, U.S. Pat. No. 8,135,227, U.S. Pat. No. 8,494,298, US20130329190, WO2008119187, EP2143273, EP2621169, CA2916780, CA2682624, US20150355481, WO2014100891, EP2939065, CA2896891, AU2013370897, US20130215147, WO2013120180, EP2814434, CA2864917, WO2013177654, EP2859399, CA2875261, US20110043644, WO201160525, EP2502410, CA2781064, US20160314564, WO2016168913, US20160282624, CA-164180F.

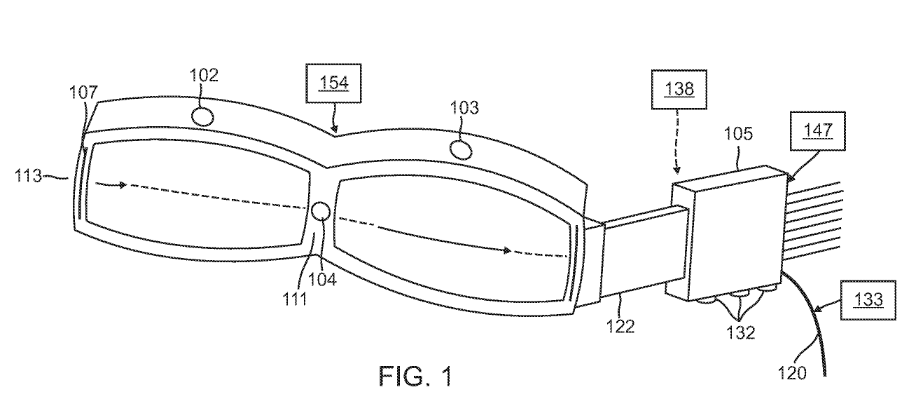

Referring now to FIG. 1-3, those skilled in the art know that hardware 111, being an exemplary—but no limiting, schematic example of wearable electronic head-mounted augment reality glasses encompasses at least one video camera 103/102/104, each for presenting imagery to a wearer/user centered on a user's macula, as described herein and in the patients incorporated expressly herein by reference.

Similarly, FIG. 1 shows computational processing device 105, in combination with 120/133/147 which are supplemental means for controlling specific computations including physical/remote and voice control and optional AI modules 138/154. Physical barrier 113 prevents external screen light from impinging on the portions of user's retina, along with functional controls 132, including buttons and counters. Device 111 provides an undiminished, naturally wide overall field-of-view limited only by the physical superstructure of the frames of the glasses themselves, readily interchanged by those of skill in the art. Likewise, those of skill understand that device 111 merely depicts schematically functional elements which may be implemented in contact lenses, IOLs, other framed assemblies and chimeric combinations of the same.

It is contemplated that the processes described above are implemented to a system configured to present an image to the user. The processes may be implemented in software, such as machine readable code or machine executable code that is stored on a memory and executed by a processor. Input signals or data is received by the unit from a user, cameras, detectors or any other device. Output is presented to the user in any manner, including a screen display or headset display. The processor and memory may be part of the headset shown in FIG. A or a separate component.

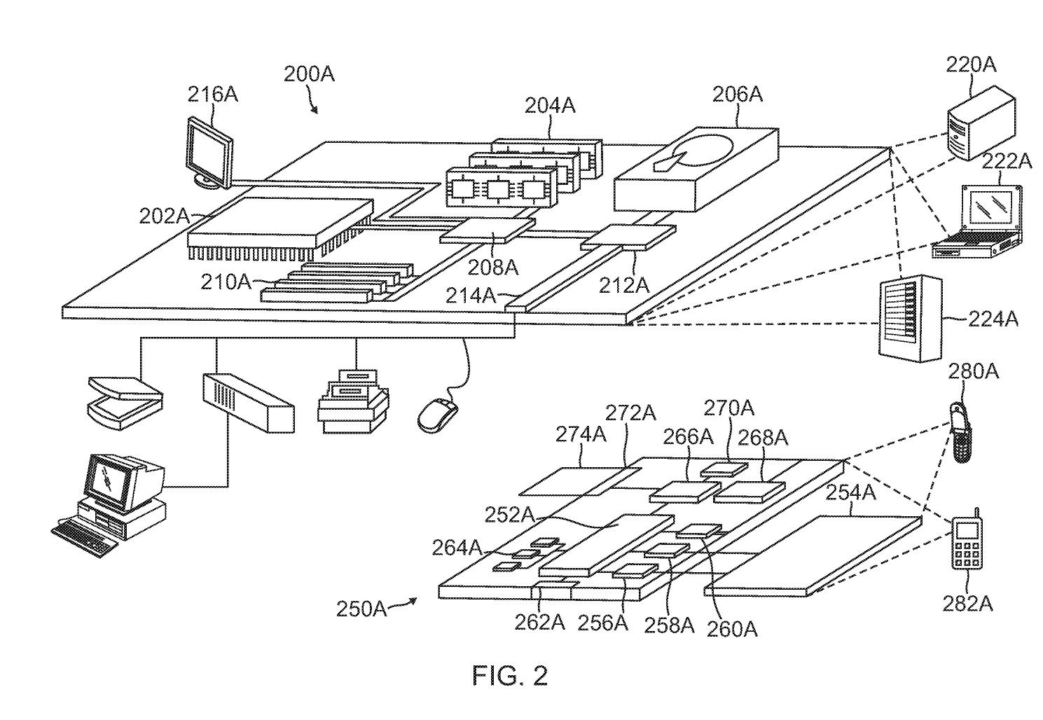

FIG. 2 is a block diagram showing example or representative computing devices and associated elements that may be used to implement the methods and serve as the apparatus described herein. Those of skill understand any of 132/120/147/154 and 105 interface with outputs and inputs from FIG. 2, which merely shows an example of a generic computing system showing one set of means for computing including for example device 200A and a generic mobile computing device 250A, which may be used with the techniques described here. Computing device 200A is intended to represent various forms of digital computers, such as laptops, desktops, workstations, personal digital assistants, servers, blade servers, mainframes, and other appropriate computers. Computing device 2S0A is intended to represent various forms of mobile devices, such as personal digital assistants, cellular telephones, smart phones, and other similar computing devices. The components shown here, their connections and relationships, and their functions, are meant to be exemplary only, and are not meant to limit implementations of the inventions described and/or claimed in this document.

The memory 204A stores information within the computing device 200A. In one implementation, the memory 204A is a volatile memory unit or units. In another implementation, the memory 204A is non-volatile memory unit or units. In another implementation, the memory 204A is a non-volatile memory unit or units. The memory 204A may also be another form of computer-readable medium, such as a magnetic or optical disk.

The storage device 206A is capable of providing mass storage for the computing device 200A. In one implementation, the storage device 206A may be or contain a computer—200A. In one implementation, the storage device 206A may be or contain a computer-reading medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 204A, the storage device 206A, or memory on processor 202A.

The high speed controller 208A manages bandwidth-intensive operations for the computing device 200A, while the low-speed controller 212A manages lower bandwidth-intensive operations. Such allocation of functions is exemplary only. In one implementation, the high-speed controller 208A is coupled to memory 204A, display 216A (e.g., through a graphics processor or accelerator), and to high-speed expansion ports 210A, which may accept various expansion cards (not shown). In the implementation, low-speed controller 212A is coupled to storage device 206A and low-speed bus 214A. The low-speed bus 214, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 200A may be implemented in a number of different forms, as shown in the figure. For example, it may be implemented as a standard server 220A, or multiple tunes in a group of such servers. It may also be implemented as part of a rack server system 224A. In addition, it may be implemented in a personal computer such as a laptop computer 222A. Alternatively, components from computing device 200A may be combined with other components in a mobile device (not shown), such as device 250A. Each of such devices may contain one or more of computing device 200A, 250A, and an entire system may be made up of multiple computing devices 200A, 250A communicating with each other.

Computing device 250A includes a processor 252A, memory 264A, an input/output device such as a display 254A, a communication interface 266A, and a transceiver 268A, along other components. The device 250A may also be provided with a storage device, such as a Microdrive or other device, to provide additional storage. Each of the components 250A, 252A, 264A, 254A, 266A, and 268A, are interconnected using various buses, and several of the components may be mounted on a common motherboard or in other manners as appropriate.

The processor 252A can execute instructions within the computing device 250A, including instructions stored in the memory 264A. The processor may be implemented as a chipset of chips that include separate and multiple analog and digital processors. The processor may provide, for example, for coordination of the other components of the device 250A, such as control of user interfaces, applications run by device 250A, and wireless communication by device 250A.

Processor 252A may communicate with a user through control interface 258A and display interface 256A coupled to a display 254A. The display 254A may be, for example, a TFT LCD (Thin-Film-Transistor Liquid Crystal Display) or an OLED (Organic Tight Emitting Diode) display, or other appropriate display technology. The display interface 256A may comprise appropriate circuitry for driving the display 254A to present graphical and other information to a user. The control interface 258A may receive commands from a user and convert them for submission to the processor 232A. In addition, an external interface 262A may be provided in communication with processor 252A, so as to enable near area communication of device 250A with other devices. External interface 262A may provide for example, for wired communication in some implementations, or for wireless communication in other implementations, and multiple interfaces may also be used.

The memory 264A stores information within the computing device 250A. The memory 264A can be implemented as one or more of a computer-readable medium or media, a volatile memory unit or units, or a non-volatile memory unit or units. Expansion memory 274A may also be provided and connected to device 250A through expansion interface 272A, which may include, for example, a SIMM (Single In Line Memory Module) card interface. Such expansion memory 274A may provide extra storage space for device 250A, or may also store applications or other information for device 250A. Specifically, expansion memory 274A may include instructions to carry out or supplement the processes described above, and may include secure information also. Thus, for example, expansion memory 274A. may be provided as a security module for device 250A, and may be programmed with instructions that permit secure use of device 250A. In addition, secure applications may be provided via the SIMM cards, along with additional information, such as placing identifying information on the SIMM card in a non-backable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 264A, expansion memory 274A, or memory on processor 252A, that may be received, for example, over transceiver 268A or external interface 262A.

Device 250A may communicate wirelessly through communication interface 266A, which may include digital signal processing circuitry where necessary. Communication interface 266A may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 268A. In addition, short-range communication may occur, such as using a Bluetooth, WI-FI, or other such transceiver (not shown). In addition, GPS (Global Positioning System) receiver module 270A may provide additional navigation- and location-related wireless data to device 250A, which may be used as appropriate by applications running on device 250.

Device 250A may also communicate audibly using audio codec 260, which may receive spoken information from a user and convert it to usable digital information. Audio codec 260A may likewise generate audible sound for a user, such as through a speaker, e.g., in a handset of device 250A. Such sound may include sound from voice telephone calls, may include recorded sound (e.g., voice messages, music files, etc.) and may also include sound generated by applications operating on device 250A.

The computing device 250A may be implemented in a number of different forms, as shown in the figure. For example, it may be implemented as a cellular telephone 280A. It may also be implemented as part of a smart phone 282A, personal digital assistant, a computer tablet, or other similar mobile device.

Thus, various implementations of the system and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive, data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback; and input from the user can be received in any form, including acoustic, speech, or tactile input.

The systems and techniques described here can be implemented in a computing system (e.g., computing device 200A and/or 250A) that includes a back end component (e.g., as a data server), or that includes a middleware component (e.g., an application sender), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network, (“WAN”) and the Internet.

The computing system can include clients and serves. A client and server are generally remote from each other and typically interact through a communication network. The relationship of client and server arises by virtue of computer programs running on the respective computers and having a client-server relationship to each other.

In the example embodiment, computing devices 200A and 250A are configured to receive and/or retrieve electronic documents from various other computing devices connected to computing devices 200A and 250A through a communication network, and store these electronic documents within at least one of memory 204A, storage device 206A, and memory 264A. Computing devices 200A and 250A are further configured to manage and organize these electronic documents within at least one of memory 204A, storage device 206A, and memory 264A using the techniques described here.

In the example embodiments computing devices 200A and 250A are configured to receive and/or retrieve electronic documents from various other computing devices connected to computing devices 200A and 250A through a communication network, and store these electronic documents within at least one of memory 204A, storage device 206A, and memory 264A. Computing devices 200A and 250A are further configured to manage and organize these electronic documents within at least one of memory 204A, storage device 206A, and memory 264A using the techniques described herein.

In addition, the logic flows depicted in the figures do not require the particular order shown, or sequential order, to achieve desirable results. Furthermore, other steps may be provided or steps may be eliminated from the described flows, and other components may be added to, or removed from, the described systems. Accordingly, other embodiments are within the scope of the following claims.

It will be appreciated that the above embodiments that have been described in particular detail are merely example or possible embodiments, and that there are many other combinations, additions, or alternatives that may be included. For example, while online gaming has been referred to throughout, other applications of the above embodiments include online or web-based applications or other cloud services.

Also, the particular naming of the components, capitalization of terms, the attributes, data structures, or any other programming or structural aspect is not mandatory or significant, and the mechanisms that implement the invention or its features may have different names, formats, or protocols. Further, the system may be implemented via a combination of hardware and software, as described, or entirely in hardware elements. Also, the particular division of functionality between the various system components described herein is merely exemplary, and not mandatory; functions performed by a single system component may instead be performed by multiple components, and functions performed by multiple components may instead be performed by a single component.

Some portions of the above description present features in terms of algorithms and symbolic representations of operations an information. These algorithmic descriptions and representations may be used by those skilled in the data processing arts to most effectively convey the substance of their work to others skilled in the art. These operations, while described functionally or logically, are understood to be implemented by computer programs. Furthermore, it has also proven convenient at times, to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “identifying” or “displaying” or “providing” or the like, refer to the action and processes of a computer system, or similar electronic computing device, that manipulates and transforms data represented as physical (electronic) quantities within the computer system memories or registers or other such information storage, transmission or display devices.

Based on the foregoing specification, the above-discussed embodiments of the invention may be implemented using computer programming or engineering techniques including computer software, firmware, hardware or any combination or subset thereof. Any such resulting program, having computer-readable and/or computer-executable instructions, may be embodied or provided within one or more computer-readable media, thereby making a computer program product i.e., an article of manufacture, according to the discussed embodiments of the invention. The computer readable media may be, for instance, a fixed (hard) drive, diskette, optical disk, magnetic tape, semiconductor memory such as read-only memory (ROM) or flash memory, etc., or any transmitting/receiving medium such as the Internet or other communication network or link. The article of manufacture containing the computer code may be made and/or used by executing the instructions directly from one medium, by copying the code from one medium to another medium, or by transmitting the code over a network.

While the disclosure has been described in terms of various specific embodiments, it will be recognized that the disclosure can be practiced with modification within the spirit and scope of the claims.

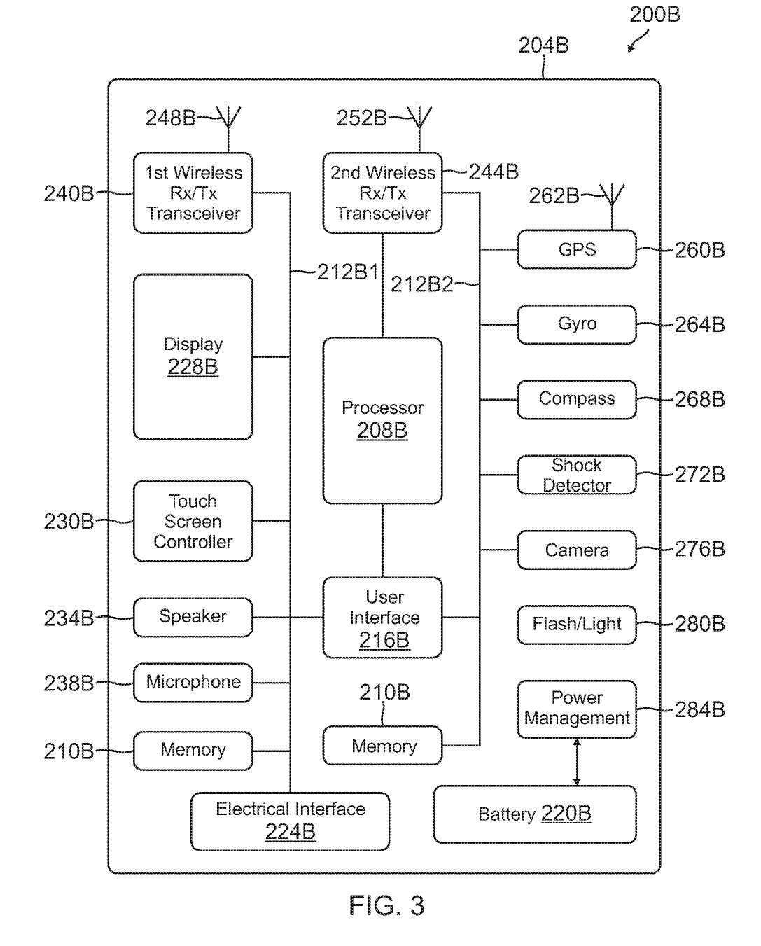

FIG. 3 illustrates an example embodiment of a mobile device 200B. This is but one possible device configuration, and as such it is contemplated that one of ordinary skill in the art may differently configure the mobile device. Many of the elements shown in FIG. 3 may be considered optional and not required for every embodiment. In addition, the configuration of the device may be any shape or design, may be wearable, or separated into different elements and components. The device 200B may comprise any type of fixed or mobile communication device that can be configured in such a way so as to function as described below. The mobile device may comprise a PDA, cellular telephone, smart phone, tablet PC, wireless electronic pad, or any other computing device.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they axe understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820.

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modern jack, HDMI port, mini-HDMI port, USB port), touchscreen (e.g., CRT, LCD, LED, AMOLED, Super AMOLED), pointing device, trackpad, light (e.g., LEO), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A wearable electronic head-mounted augmented reality (AR) glasses low vision aid comprising, in combination:
At least one embedded video camera situated and configured for capturing real-time imagery that encompasses at least the field-of-view of a normally-sighted human wearer;
an embedded display or displays, each presenting imagery directly to one of the wearer's eyes—
said imagery originating from said embedded camera(s)
said imagery processed, enhanced, or manipulated arbitrarily according to the user's need or benefit, including magnification and nonlinear transformations,
said display(s) arranged such that their contents will be centered upon the macula of the retina of the associated eye, where the highest visual acuity would be available to a normally-sighted wearer;
at least a computational processing device, embedded or remote, for producing processed said images for said display(s) from camera images;
a means for controlling the specific computations performed by said computational device, including physical control surfaces, remote-control devices, voice control, or autonomous software-based decision; and,
a physical barrier, placed explicitly or existing implicitly in the construction of the display, that prevents external scene light (especially light originating directly behind the apparent position of the. displays) from impinging on fee same portions of the retina as imagery from the displays,
said barrier eliminating focus ambiguities that confound low-vision users, whereby unobstructed pathways for external scene light to enter the eye(s) and impinge on the retina directly around the periphery (sides and preferably top and bottom) of the displayed image(s) at the natural retinal focus (as if no glasses were present for said rays of external scene light),
said unobstructed pathways providing failsafe vision if display devices fail to function, and
said unobstructed pathways providing a zero-latency reference for equilibrium.

2. The wearable electronic head-mounted augmented reality (AR) glasses low vision aid of claim 1, further comprising additional processing and computations (using the computational processing device) upon displayed image(s) such that the boundary between natural scene lighting and displayed imagery upon the retina can appear natural and seamless when interpreted by the wearer's brain; said natural-seeming transition maintaining even when arbitrary non-natural processing is applied near the center of the displayed image(s).

3. The wearable electronic head-mounted augmented reality (AR) glasses low vision aid of claim 2, further comprising output providing an undiminished, naturally-wide overall field-of-view limited only by the physical superstructure of the glasses themselves, even if the active display field-of-view is much smaller.

4. An improved model for representing any two-dimensional monotonic mapping with arbitrary accuracy comprising, in combination:
a. a two-dimensional source space
b. a two-dimensional destination space
c. a rectangular input domain describing the boundaries of the source space
d. the rectangular input domain characterized as a rectangle using its length, width, and lower-left corner
e. a partitioning of the input domain into a uniform grid of smaller rectangles
i. said partitioning characterized by its resolution, the number of smaller rectangles in each axis
ii. said resolution being arbitrary, with as few as one rectangle per axis
f. a mapping from each point on the uniform grid office input domain to the destination space
g. a continuous underlying mathematical model for the number of points lying between the defined uniform grid locations of the input space, and
h. At least an algorithm for computing the mapping from any coordinates in the source space to their corresponding coordinates in the destination space, regardless of whether the source coordinate lie on the uniform grid of the input domain.

5. The improved model of claim 4, further comprising a B-spline basis used for the continuous underlying mathematical model.

6. The improved model of claim 5, with a uniformly-spaced B-spline basis as the continuous underlying mathematical model.

7. The improved model of claim 6, being a scalable model for representing two-dimensional monotonic mappings with changeable resolution, comprising the above model using uniformly-spaced B-spline basis additionally coupled with:
an algorithm for increasing the resolution of the model in one or both dimensions, without perturbing the existing mapping, and
an algorithm for decreasing the resolution of the model in one or both dimensions, with minimal impact to the existing mapping.

8. The improved model of claim 7, with the algorithm for increasing model resolution being a separable (i.e. per-axis) one-dimension 2× up-sampling operator applied independently to each dimension.

9. The improved model of claim 8, with the algorithm for decreasing model resolution being a separable (i.e. per-axis) one-dimension 2× down-sampling (i.e. subspace projection) operator applied independently to each dimension.

10. The improved model of claim 9, with the above algorithm for increasing model resolution based on separable 2× up-sampling and the algorithm for decreasing model resolution based on separable 2× down-sampling, extended such that the source space is extended by one additional grid point in each of the four directions, with the new grid points having the following characteristics:
said additional grid points being constrained to remain in their natural position i.e. unaffected by the mapping) regardless of any changes or editing that occur to other grid points;
i. an inverse model for any designated two-dimensional monotonic mapping (i.e., the “forward model”), said inverse model itself being a two-dimensional monotonic mapping with its source space chosen as a rectangular subset of the destination space of the designated two-dimensional mapping destination, and with its destination space being the source space of the designated two-dimensional mapping;
ii. said inverse model with its destination space corresponding coinciding with the boundaries of a display device, and with a resolution of exactly one pixel in each dimension for efficient usage.

11. An algorithm for efficiently deriving an inverse model from its forward model, comprising, in combination:
an iterative two-dimensional search evaluated at each uniform grid point in the inverse model; and
a schedule for the order of uniform grid points considered in the inverse model, chosen such that the intermediate computations and results for the previous point help bootstrap the iterative search for the next point in order to speed convergence with minimum iterations and computations.

12. A method for efficiently performing the backward transform on a sampled digital image at high rate capable of supporting real-time video sources, comprising:
a model (scalable or otherwise) for the forward mapping;
an algorithm for deriving an inverse model from its forward model;
an inverse model computed using said algorithm, with its destination space; corresponding coinciding with the boundaries of a display device, and with a resolution of exactly one pixel in each dimension;
said inverse model computed ONLY when any portion of the corresponding forward mapping changes;
said inverse model converted to a lookup table;
said lookup table used in a Graphic Processing Unit (GPU) shader to perform the mapping at speeds supporting real-time display
the above method of efficiently implementing the backward transform for real-time display, where the forward model uses a Bspline mathematical basis such that the inverse model only requires local updates rather than a full recomputation when a portion of the forward mapping changes, in combination being a method for layering, or pipelining multiple models and algorithms described above in order to produce multiple effects in an efficient manner.

13. A method for interactively displaying images with a mapping applied (i.e. moving visual information from one location to another within a visual field), comprising, in combination:
a. a display device for showing the final mapped image,
b. an image source for providing the initial image,
c. a mapping, from one of the types described above
d. a processor that computes the final mapped image from the initial image
e. the method above, for interactively displaying images with a mapping applied, where the display device is a wearable augmented-reality glasses, and the processor is embedded in the glasses
f. the method above, for interactively displaying images with a mapping applied, where the display device is a wearable augmented-reality glasses, the processor is embedded in the glasses, and the mapping is a two-dimensional model with underlying continuous uniform B-spline basis.
g. the method above, for interactively displaying images with a mapping applied, where the display device is a wearable augmented-reality glasses, the processor is embedded in the glasses, and the mapping is an efficient inverse model derived (as in an above claim) from an underlying forward model with underlying continuous uniform B-spline basis.
h. a method for displaying layered, or pipelined multiple models and algorithms described above in order to produce multiple effects in an efficient manner.

14. A method for interactively revealing or characterizing qualitative aspects of visual field distortion defects including:
a. a display device maintaining a fixed physical orientation with respect to the viewer's eye, such that the viewer can comfortably fixate and maintain his gaze upon the center of the display
b. said display presenting a controllable image to the wearer
c. said controllable image comprising a regular reference grid or reference lines superimposed on a background
d. said reference grid having variable grid spacing in each dimension, and variable phasing (offset) in each dimension with respect to the boundary of the display
e. said reference lines being one or more parallel and/or perpendicular lines, each parallel, to one of the display axes, with variable positions on the display
f. said reference grid or reference lines having highly-visible coloration and brightness
g. said background comprising one of the following;
i. a featureless uniform black background
ii. a movable but otherwise static image containing finely-detailed structures (such as text or icons)
iii. the above static image with subdued brightness and coloration
iv. a video feed from a live camera or stored video source
v. the above video feed with subdued brightness and coloration
vi. a combination of video feed with superimposed static imagery
vii. a control interface for configuring the specific characteristics of the reference grid, reference lines, and background, comprising one or more of the following:
physical joysticks
physical buttons
standard or customized keyboards or keypads
wireless handheld remote control devices
applications running on mobile phones, tablets, or computing devices
applications running within a web browser
position and movement sensors
speech recognition
finger or object tracking.

15. The method of claim 14, where the display device is a wearable optical-see-through (OST) augmented reality (AR) glasses such that the drawn reference grid, reference lines, or static image details are automatically superimposed upon the user's natural view of the world due to their construction (not requiring any computational effort or processing)
a. the above method, where the display device is a wearable augmented reality (AR) glasses that does NOT provide an OST path coinciding with the extent of the display image, but where a background image is instead provided by a live camera feed that is slaved to the wearer's head and approximating the view that would be seen by the user if the glasses were not being worn.
b. the above methods combined, where a control interface is available to the wearer to permit self-actuated, unconstrained, interactive exploration of the effects of distortion as the characteristics of the reference grid and/or reference lines and/or background contents are changed
c. combining the above methods by layering, or pipelining multiple models and algorithms described above in order to produce multiple effects in an efficient manner, which are interactively controlled by the user.

16. A method for interactively characterizing and correcting visual field distortion defects using a multi-resolution model and structured hierarchical editing, comprising:
a. a forward model for the mapping from the physical display image coordinates to the perceived image coordinates available only within the mind of the wearer
b. said forward model initialized to an initial forward model having the lowest possible internal grid resolution (i.e. a single rectangle with four grid points) and indicating an identity transformation (i.e. no distortion being present)
c. an inverse model corresponding to the forward model
d. said inverse model being maintained concurrently with the forward model, and updated as necessary whenever the forward model is changed
e. the above method for interactively characterizing visual field distortion detects, augmented such that the image can optionally have the inverse model applied to it before being displayed, such that if a the forward model were a perfect representation of the true distortion mapping from displayed image to perceived image, the resulting perceived image would no longer be distorted.
f. at least two distinct operating modes for the device, comprising:
i. a normal operating mode, in which distortions are optionally are corrected using an inverse model
ii. an editing mode, wherein the user can interactively explore, characterize, and correct his perceived distortion by making structured changes to the model
g. the control interface being available directly to the user, and providing the following capabilities while in editing mode:
i. all abilities attributed to the method for interactively characterizing visual field distortions
ii. an additional grid manipulation mode wherein the specific grid corresponding to the forward model is displayed, and the optional ability to apply the diverse model to the displayed image is also active in order to allow the user to evaluate the verisimilitude of the model,
iii. an interface to narrow the scope of the forward model such that it isolates only a limited rectangular subset of the total display to indicate the extents of regions where the user perceives distortion
iv. an interface method to select a grid point such that it is clearly displayed as being the currently selected point
v. an interface method to move the currently selected grid point around
vi. an interface method to de-select the currently selected grid point, leaving it at its current position
vii. an interface method to remove any changes to the currently selected grid point, returning it to the position it occupied before it was last selected
viii. an interface method to undo some number of immediately recent changes to grid point locations
ix. an interface method to increase the resolution of the model without changing net mathematical mappings, but simply resulting in a higher density of grid points available for manipulation
x. an interface method to decrease the resolution of the model such that a lower density of grid points is available for manipulation
xi. an interface to accept the current model and cease editing
xii. an interface to begin incremental editing with the current model as a starting point
xiii. an interface to begin editing with a new initial forward model as a starting point
h. the above mentioned control interface method to drag a grid point causing the underlying forward model to be adjusted in a corresponding fashion in real time, as the wearer directly manipulates the model interactively change the local appearance of the distortion in such a way as to ameliorate its effects
i. an internal regulatory process to constrain the dragging of grid points so as to maintain the property that models remain monotonic, i.e. by preventing the dragged point from crossing the line segments that are not attached to it.
j. optional internal regulatory processes that regularize the model, smoothing it either automatically, upon request, or at key checkpoints (when model resolution changes, or
the model is finalized)
k. the interactive method described above, but with its initial model bootstrapped with a-priori model information obtained using external means such as visual field distortion measurement via Amsler Grid or other standardized assessment technique
l. the method above for interactively characterizing and correcting visual field distortion defects, implemented on wearable AR glasses as a self-contained system with embedded display or displays, embedded processor performing all computations, and the forward model begin based on an underlying uniform B-spline basis with efficient inverse models being updated incrementally and realized as efficient GPU shaders.

17. The method of claim 16, for interactively characterizing and correcting visual field distortion defects, implemented on wearable AR glasses as a self-contained system with embedded display or displays, embedded processor performing all computations, and the forward model
being scalable and based on an underlying uniform B-spline basis with efficient inverse models being updated incrementally and realized as efficient GPU shaders.

18. The method of claim 17, which further comprises the steps of:
including a finger-tracking AR control interface wherein the user selects and manipulates grid points during editing mode by interacting with those grid points as virtual objects; and
including the ability to layer, or pipeline multiple methods, models and algorithms in order to produce multiple effects in an efficient manner, which are interactively controlled by the user.

19. Devices, according to the method of claim 17, not implemented on wearable AR glasses as a self-contained system with embedded display or displays, rather in contact lenses, Intra-ocular lenses, frames of same manner and/or Chimeric combinations of the same.

20. Devices, for visually challenged users, embodying the method of claim 13.
